(19) United States
(12) Patent Application Publication
(10) Pub. No.: US 2014/0336510 A1
(43) Pub. Date: Nov. 13, 2014

(54) Enhancement in Diagnostic Ultrasound Spectral Doppler Imaging

(71) Applicants: Mikyoung Park, Issaquah, WA (US); Paul Donald Freiburger, Seattle, WA (US)

(72) Inventors: Mikyoung Park, Issaquah, WA (US); Paul Donald Freiburger, Seattle, WA (US)

(73) Assignee: Siemens Medical Solutions USA, Inc., Malvern, PA (US)

(21) Appl. No.: 13/890,171

(22) Filed: May 8, 2013

(51) Int. Cl. A61B 8/08; A61B 8/14; A61B 8/00

(52) U.S. Cl. CPC A61B 8/5269; A61B 8/463; A61B 8/14; A61B 8/5207
USPC 600/441

(57) ABSTRACT

Spectral Doppler imaging is enhanced. The boundary between noise and signal is determined in each spectrum. The boundary is used to differentiate noise from signal. The noise level is reduced and/or the signal level is increased in the respective regions of the spectrum, providing more distinguishable regions.

### BACKGROUND

The present invention relates to spectral Doppler ultrasound. By transmitting a plurality of pulses (pulsed wave) or a continuous wave at a single gate location, a spectral Doppler response is generated in response to received echo signals. The frequency spectrum of the object's motion or flow for a single spatial region is estimated and displayed as a function of time. Spectral Doppler ultrasound imaging provides an image of spectra as velocity (vertical axis) values modulated by energy as a function of time (horizontal axis) for a gate location. The spectra may be used for studying fluid flow or tissue motion within a patient.

Spectral boundaries are identified in the spectra to assist in diagnosis. The spectral boundary may indicate a maximum or minimum velocity of flow or tissue motion over time. However, the spectral boundary may be unclear to a user due to poor signal-to-noise ratio (SNR).

The SNR of spectral Doppler is poor in certain examinations. For example, the SNR is poor in thyroid and renal examinations. FIG. 1 shows a spectral Doppler image with poor SNR. The boundary between velocities associated with signal and velocities associated with noise is around the 200 level in the time range of 0-5. The boundary may be distinguished by the change in the level of energy modulation, shown as a difference in gray level. The boundary is non-distinct. The lack of distinction between noise and signal may make it difficult to see spectral boundaries clearly.

### BRIEF SUMMARY

By way of introduction, the preferred embodiments described below include methods, systems, computer readable media, and instructions for enhancing spectral Doppler imaging. The boundary between noise and signal is determined in each spectrum. The boundary is used to differentiate the noise from the signal. The noise level is reduced and/or the signal level is increased in the respective regions of the spectrum, providing more distinguishable regions.

In a first aspect, a method is provided for enhancing spectral Doppler imaging. A spectrum is estimated for a Doppler gate location. A boundary between a noise region and a signal region of the spectrum is detected. A processor alters the noise region, the signal region, or the noise and signal region of the spectrum. The altering is a function of the boundary. An image is displayed. The image is a function of the altered spectrum.

In a second aspect, a non-transitory computer readable storage medium has stored therein data representing instructions executable by a programmed processor for enhancing spectral Doppler imaging. The storage medium includes instructions for locating an envelope in a spectral strip, increasing a separation between signals of the spectral strip on different sides of the envelope, and displaying the spectral strip with the signals having the increased separation.

In a third aspect, a system is provided for enhancing spectral Doppler imaging. A transmit beamformer is operable to transmit acoustic energy to a Doppler gate. A receive beamformer is operable to sample acoustic echoes from the Doppler gate and in response to the acoustic energy. A spectral Doppler processor is configured to estimate a spectrum from the samples of the acoustic echoes for the Doppler gate. A second processor is configured to detect noise and signal regions of the spectrum and to weight the noise and signal regions of the spectrum differently.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is an example spectral Doppler image;

FIG. 2 is a flow chart diagram of one embodiment of a method for enhancing spectral Doppler imaging;

FIG. 3 is a graphical representation of an example spectrum;

FIG. 4 is a graphical representation of an example spectrum with velocity modulated by energy mapped to a y-axis for a time and an example boundary;

FIG. 5 is a graphical representation of an example ramped scale function;

FIG. 6 is an example enhanced spectral Doppler image; and

FIG. 7 is a block diagram of one embodiment of a system for enhancing spectral Doppler imaging.

### DETAILED DESCRIPTION OF THE DRAWINGS AND PRESENTLY PREFERRED EMBODIMENTS

The signal-to-noise ratio (SNR) is enhanced in spectral Doppler imaging. The SNR is improved by identifying boundaries between signal regions and noise regions and then attenuating the noise region and/or enhancing the signal region.

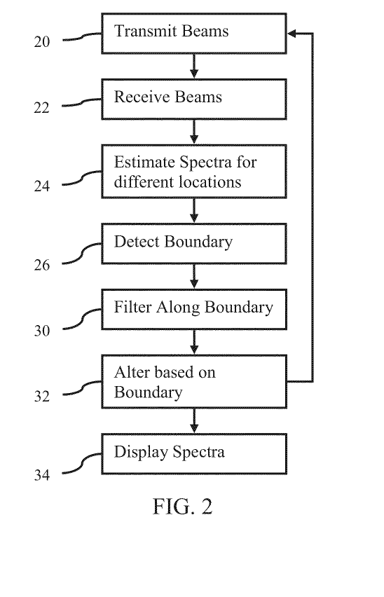

FIG. 2 shows method for enhancing spectral Doppler imaging. The method is implemented on the system 10 of FIG. 7 or a different system. A processor controls and/or performs the acts. One or more acts may be performed through interaction with a user. Other acts or all the acts may be performed automatically by a processor without user input other than activation or gate location determination.

The acts are performed in the order shown, but other orders are possible. Additional, different, or fewer acts may be provided. For example, act 30 is not performed. As another example, act 34 is not performed. In yet another example, acts for filtering, processing, or other spectral Doppler functions are provided.

The method is implemented for pulsed wave (PW) or continuous wave (CW) spectral Doppler. “Doppler” is used to express spectral processing in general. Other spectral processes using ultrasound samples from different times may be used. In PW, a gate location is sampled using pulse wave (e.g., 1-10 cycles) transmissions interleaved with echo reception. PW may interleave with other modes of imaging, such as B-mode or flow-mode. In CW, a continuous wave (e.g., hundreds or thousands of cycles) is transmitted to the gate location, and echoes are received while transmitting.

For spectral Doppler imaging, the sample gate or spectral Doppler gate is positioned. For example, a B-mode and/or flow-mode scan is performed. The user indicates a gate location on the resulting image. In other examples, the gate is automatically positioned, such as at a location of greatest Doppler velocity or energy determined from flow-mode data.

In act 20, a plurality of beams of acoustic energy is transmitted. The acoustic energy of each transmission is focused at or near the gate location. The focus results in generation of a transmit beam.

The transmissions are repeated. The repetition allows reception of sufficient samples to perform spectral analysis.

In act 22, acoustic echoes are received. The echoes are received in response to the transmission of the acoustic energy. The echoes are sampled to acquire received signals at the gate location. Receive beams are formed by focusing the received signals to coherently combine data representing the gate location.

The receive operation occurs repetitively in response to the repetitive transmissions. Beamformed samples from the gate location at different times are received. Samples for the same location are acquired over time. For spectral analysis, an ensemble of samples from a same location is acquired, such as five to twenty samples for each spectrum. The samples may be obtained in an ongoing manner such that a moving window (e.g., ensemble or flow sample count) with any step size (e.g., every sample or every third sample) is used to estimate a spectrum. Any scan sequence and/or pulse repetition frequency may be used.

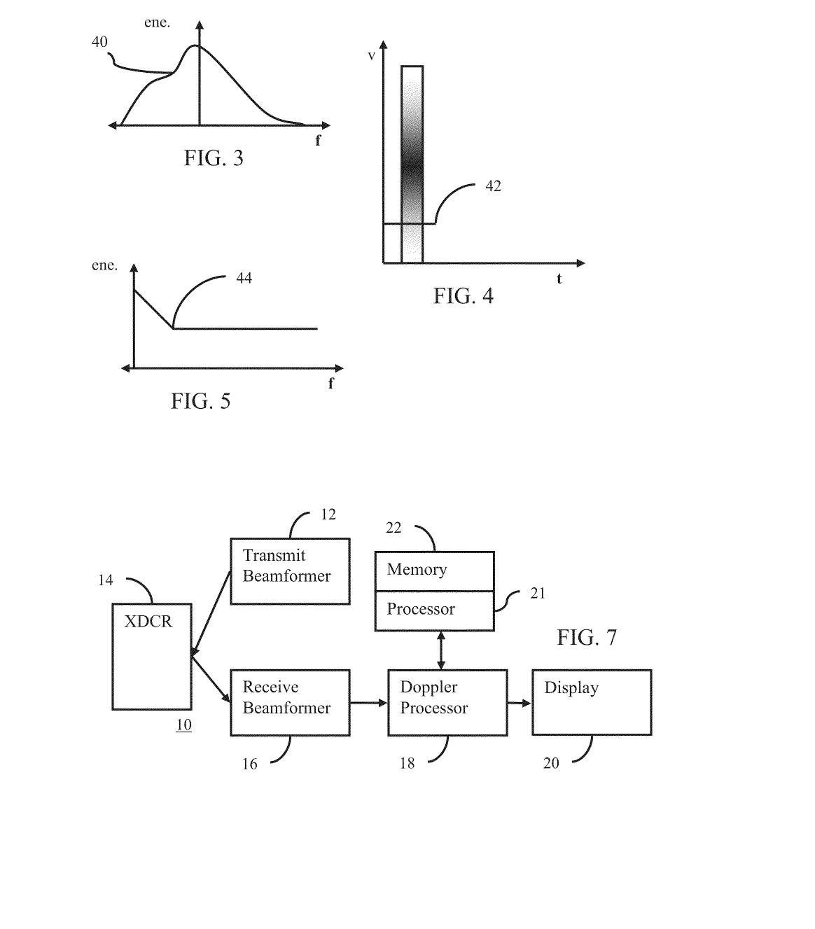

In act 24, a spectrum is estimated for the Doppler gate location. The spectrum is estimated by applying a Fourier transform, wavelet transform, or Wigner-Ville distribution to the sequence of ultrasound samples. Any transform may be applied to determine the spectrum. As shown in FIG. 3, the spectrum 40 represents energy as a function of frequency. Frequency has a known relationship to velocity, so expression in terms of frequency provides velocity and expression in terms of velocity provides frequency.

The spectrum is estimated from the ultrasound samples in the sequence from the Doppler gate location. The spectrum corresponds to a period in which the samples were acquired. The spectrum represents a time or the period. A sequence of spectra represents the Doppler gate location at different times. Other spectra may be estimated for other periods or different times corresponding to different periods or ensembles of acquisition. The periods may overlap, such as when using a moving window with a step size less than the ensemble period, or may be unique.

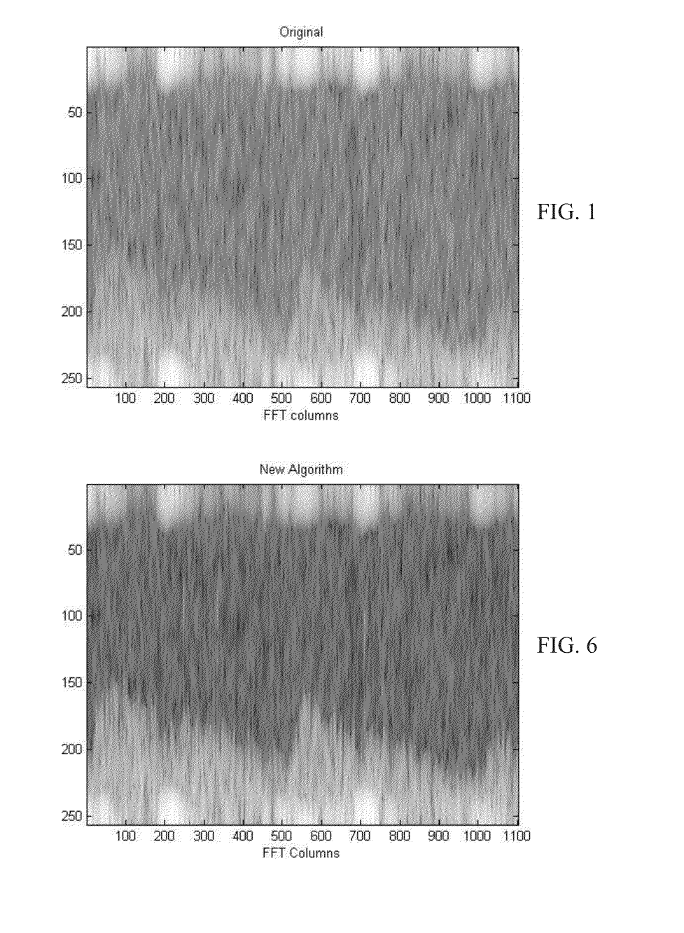

FIG. 1 shows a spectral strip of spectra for a same location over time. The spectrum for a given time in a spectral strip is mapped with velocity on the horizontal axis and energy modulating the intensity, as shown by the graphical representation of the spectrum of FIG. 4. Other mapping may be used.

In act 26, a boundary between a noise region and a signal region of the spectrum is detected. The signal region corresponds to velocities with greater energy. For example, the velocities with darker modulation in the examples of FIG. 4 have greater energy. A maximum and/or minimum velocity of the tissue or fluid is at the boundary with the noise region. In the noise region, the energy may vary, but is generally below the level of the signal region. Similarly, in the signal region, the energy may vary but is generally above the level of the noise region.

Greater SNR provides greater distinction. FIG. 4 shows a boundary 42 below which is a noise region and above which is a signal region. The boundary is at a velocity or velocities determined to be from signal adjacent to a velocity or velocities determined to be from noise. The boundary may be set at a velocity bin or value associated with noise or signal, depending on the boundary detection technique. The signal region may extend to the lowest or highest velocity or may have a lesser extent such that another two boundaries exist whether or not detected.

The boundary is detected using any technique, such as using a waveform trace. A user may manually indicate the boundary. A processor may use clutter measures. For example, clutter may be measured based on mapping from velocity and energy, such as high energy with low velocity indicating stronger clutter strength. Clutter may be measured by a ratio or difference of energy with and without clutter filtering. A processor may apply an energy threshold. For example, the greatest and/or lowest velocity with one, two, three, or other number of consecutive velocities having energies above a predetermined threshold is identified as the boundary. The threshold may adapt, such as measuring an average energy of noise in the system or imaging situation and setting the threshold to a level above the average (e.g., 10% increase). The noise may be measured as energy or brightness of the spectrum or spectra from samples with the transmitter turned off.

The boundary is detected from the spectrum. For example, the threshold is applied to the data of the spectrum. In the spectra, an envelope separating noise from signal is determined by locating the boundary for each spectrum. In other embodiments, information from multiple spectra are used to determine the envelope location in a given spectrum. The boundary may be derived from one spectrum after comparison with other spectra for the location. For example, clutter from another spectrum is used to locate the boundary. As another example, the combination of spectra is used for pattern matching a template of spectra with the spectra for the location. The pattern indicates the boundary.

In act 30, the boundary is filtered. Where boundary detection is repeated for different spectra, the velocity location of the boundary varies over time. This envelope or boundary over time may be filtered across time. The boundary is smoothed to reduce variance over time and/or reduce spurious artifacts. The filtering occurs after detecting the boundaries for multiple spectra and before the altering. By filtering the boundary, the boundaries for individual spectra may or may not shift along the velocity scale. Once established, the filtered boundaries are used for altering in act 32. Without filtering, the detected boundaries are used.

The boundary is used to increase a separation between signals on different sides of the envelope in act 32. The apparent SNR is increased. A processor alters the spectrum or information of the spectrum to increase the separation.

The alteration is a function of the boundary. The boundary indicates what data to change and/or in what way to change. For example, the apparent SNR may be improved by filtering or averaging the noise in the identified noise region to smooth the noise out and reduce the variability. Any filtering or changes to the signal are performed differently and/or result in an apparent overall increase relative to the noise. The signal region is processed separately from the noise region, such as not using data from the signal region in the processing of the noise region and vice versa.

The separation is increased by increasing signals on the signal side of the envelope, decreasing signals on a noise side of the envelope, or both. The noise region, the signal region, or both regions are altered. The alteration for the noise region is different from the alteration for the signal region. To increase the separation, the noise is reduced and/or the signal is increased. Alternatively, other processing may increase the separation.

Any aspect of the spectrum may be altered. In one embodiment, energies are changed. For example, the energies in the noise region are attenuated. As another example, energies in the signal region are amplified. The attenuation and/or amplification are applied to all of the signals or a sub-set of the signals of the appropriate region.

In one embodiment, the energies are changed by applying a scaling factor. Noise can be attenuated or signals may be enhanced by simply applying a scaling factor (or factors) to either or both regions. For noise, the scaling factor is less than one and multiplied with the energy. For signal, the scaling factor is greater than one and multiplied with the energy. The scaling is performed differently on the different sides of the boundary, such as using different scales or applying a scale to only one region. Other functions, such as subtraction, addition, or division using the scaling function may be provided.

The scaling factor is programmable or fixed. Different scaling factors may be used for different applications. In other embodiments, the scaling factor is adaptive, such as being based on the noise level, signal level, or other feedback.

The same scaling factor is applied to a respective region. For example, all of the energies in the signal region are multiplied by 1.2 and/or all of the energies in the noise region are multiplied by 0.5. Any scaling factor may be used.

In an alternative embodiment, the scaling factor varies within the region. For example, a ramp function is used. A ramp may be applied to smooth the transition from signal to background noise. A variable scaling factor is applied across the noise and/or signal regions. For example, near the boundary, the scaling factor is ramped down for the noise to avoid a sharp discontinuity. Alternatively or additionally, near the boundary the scaling factor in the signal region is ramped up for the signal to avoid a sharp discontinuity. The variation may sharpen the high velocity profile in the signal and improve aesthetics.

FIG. 5 shows an example function 44 of a linear ramp with a floor. The function 44 is positioned spatially relative to the velocities. Noise near the boundary is not attenuated as much as noise away from the boundary. The ramp has any slope, such as extending over 5-20 velocity bins in a 0-255 velocity scale and from a 0.95 scale factor to 0.4 scale factor. Any number of bins (i.e., velocity range), slope, or number of scale factors (i.e., scale factor range) may be used. For velocities beyond the ramp, if any, a same scale factor is applied to the respective energies.

For noise attenuation, the highest scaling factor of the ramp is positioned at or over the boundary. In other embodiments, the ramp may incorporate enhancements as well such that a scale factor of 1.0 is used at the velocity of the border with increasing scale factors in the signal region and decreasing scale factors in the noise region. Other scale factors than 1.0 may be used at the boundary, such as attenuating some signal near the boundary.

Ramps or other scale factor variation may be used for the signal region. Other scale factor functions may be used for either or both regions, such as a non-linear function. Different weights and/or variation functions are applied to energies on the different sides of the boundary.

The feedback from act 32 to act 20 represents repeating the acquisition of samples, estimating for a different period, detecting the boundary for this different time, and altering the spectrum based on the boundary. For a spectral strip, the process and corresponding repetition is on-going or occurs multiple times. The boundary for each spectrum is independently determined, but may be determined based on boundaries and/or spectra from other times.

In act 34, an image is displayed. The image is a function of the altered spectrum. The altered spectrum or series of altered spectra may be used to determine a characteristic of the displayed image. For example, an icon or value representing a maximum velocity or SNR is displayed.

The altered spectrum or series of altered spectra may be used to generate a spectral strip. The spectral strip is displayed for the Doppler gate. The spectra used in the spectral strip are the altered spectra or altered energies for the spectra. Filtering may be applied to smooth the spectra. The spectral strip shows the frequency modulated by energy as a function of time. Any now known or later developed spectral strip mapping may be used, such as gray scale mapping with the intensity representing energy. The energies, after alteration, modulate the pixels. The gray scale or color is mapped from the energy values. The displayed image may be a function of a single spectrum or of multiple spectra.

Since the separation of signal from noise is increased, the resulting spectral strip has more apparent signal regions. The signals of the spectral strip have increased separation, particularly about the boundary. For example, FIG. 6 shows the spectral strip of FIG. 1, but mapped from altered energy values. Spectral Doppler results show improved SNR and/or a clearer waveform boundary.

The boundary is more visible without a graphic overlay due to the alteration. A graphic indicating the boundary may be included. Characteristics of the spectral strip or spectra may be determined and displayed, such as graphically tracking a maximum velocity as a function of time in the spectral strip.

Multiple strips may be displayed. For example, spectral strips for two or more selected locations are output for comparison. The resulting multiple spectral strips provide spectra for the desired feature of the patient.

In one embodiment, the spectral strip is displayed with a spatial image, such as a one-dimensional M-mode, two-dimensional B-mode, two-dimensional F-mode, or combination thereof image. The image is of the region of interest using data acquired for pulsed wave Doppler sampling (e.g., flow mode). The location of the gate may be indicated graphically in the image, such as represented by a circle in the region of interest of the field of view. For example, text, color, symbol, or other indicator shows the user the location for the range gate corresponding to the selected spectrum. Where multiple spectra are displayed, matched color coding between the acquisition range gates and displayed spectra may be used. Other indications may be used, such as text labels or numbering.

FIG. 7 shows a system 10 for enhancing spectral Doppler imaging. The system 10 is a medical diagnostic ultrasound imaging system, but other imaging systems may be used, such as a workstation. The system 10 estimates a spectrum or alters the spectrum or spectra for increased SNR.

The system 10 includes a transmit beamformer 12, a transducer 14, a receive beamformer 16, a Doppler processor 18, a display 20, a processor 21, and a memory 22. Additional, different or fewer components may be provided, such as the system 10 without the front-end beamformers 12, 16 and transducer 14 or the system 10 with a scan converter. The Doppler processor 18 and processor 21 may be combined into one device acting as both processors 18, 21.

The transducer 14 is an array of a plurality of elements. The elements are piezoelectric or capacitive membrane elements. The array is configured as a one-dimensional array, a two-dimensional array, a 1.5D array, a 1.25D array, a 1.75D array, an annular array, a multidimensional array, combinations thereof, or any other now known or later developed array. The transducer elements transduce between acoustic and electric energies. The transducer 14 connects with the transmit beamformer 12 and the receive beamformer 16 through a transmit/receive switch, but separate connections may be used in other embodiments.

The transmit beamformer 12 is shown separately from the receive beamformer 16. Alternatively, the transmit and receive beamformers 12, 16 may be provided with some or all components in common. Operating together or alone, the transmit and receive beamformers 12, 16 form beams of acoustic energy for sampling a gate location and/or scanning a one, two, or three-dimensional region.

The transmit beamformer 12 is a processor, delay, filter, waveform generator, memory, phase rotator, digital-to-analog converter, amplifier, combinations thereof, or any other now known or later developed transmit beamformer components. In one embodiment, the transmit beamformer 12 digitally generates transmit waveform envelope samples. Using filtering, delays, phase rotation, digital-to-analog conversion and amplification, the desired transmit waveform is generated. In other embodiments, the transmit beamformer 12 includes switching pulsers or waveform memories storing the waveforms to be transmitted. Other transmit beamformers 12 may be used.

The transmit beamformer 12 is configured as a plurality of channels for generating electrical signals of a transmit waveform for each element of a transmit aperture on the transducer 14. The waveforms are unipolar, bipolar, stepped, sinusoidal, or other waveforms of a desired center frequency or frequency band with one, multiple, or fractional number of cycles. The waveforms have relative delay and/or phasing and amplitude for focusing the acoustic energy. The transmit beamformer 12 includes a controller for altering an aperture (e.g. the number of active elements), an apodization profile (e.g., type or center of mass) across the plurality of channels, a delay profile across the plurality of channels, a phase profile across the plurality of channels, center frequency, frequency band, waveform shape, number of cycles, coding, or combinations thereof.

The transmit beamformer 12 is operable to transmit a sequence of transmit beams of ultrasound energy. A transmit beam originates from the transducer 14 at a location in the transmit aperture. The transmit beam is formed along a scan line at any desired angle. The acoustic energy is focused at a point along the scan line, but multiple points, line focus, no focus, or other spread may be used. The acoustic energy is focused at the Doppler gate location, but may be focused elsewhere (e.g., the Doppler gate is along the scan line but not at the focus). The beam of acoustic energy is transmitted to the Doppler gate.

The receive beamformer 16 is a preamplifier, filter, phase rotator, delay, summer, base band filter, processor, buffers, memory, combinations thereof, or other now known or later developed receive beamformer component. Analog or digital receive beamformers capable of receiving one or more beams in response to a transmit event may be used.

The receive beamformer 16 is configured into a plurality of channels for receiving electrical signals representing echoes or acoustic energy impinging on the elements of the transducer 14. A channel from each of the elements of the receive aperture within the transducer 14 connects to an amplifier for applying apodization amplification. An analog-to-digital converter may digitize the amplified echo signal. The radio frequency received data is demodulated to a base band frequency. Any receive delays, such as dynamic receive delays, and/or phase rotations are then applied by the amplifier and/or delay. A digital or analog summer combines data from different channels of the receive aperture to form one or a plurality of receive beams. The summer is a single summer or cascaded summer. The summer sums the relatively delayed and apodized channel information together to form a beam. Beamformed samples of echoes from the gate location are obtained.

In one embodiment, the beamform summer is operable to sum in-phase and quadrature channel data in a complex manner such that phase information is maintained for the formed beam. Alternatively, the beamform summer sums data amplitudes or intensities without maintaining the phase information. Other receive beamformation may be provided, such as with demodulation to an intermediate frequency band and/or analog-to-digital conversion at a different part of the channel.

Beamforming parameters including a receive aperture (e.g., the number of elements and which elements used for receive processing), the apodization profile, a delay profile, a phase profile, imaging frequency, inverse coding, or combinations thereof are applied to the receive signals for receive beamforming. For example, relative delays and amplitudes or apodization focus the acoustic energy along one or more scan lines. A control processor controls the various beamforming parameters for receive beamformation.

One or more receive beams are generated at the Doppler gate and in response to each transmit beam. Acoustic echoes are received by the transducer 14 in response to the transmitted acoustic energy. The echoes are converted into electrical signals by the transducer 14, and the receive beamformer 16 forms the receive beams from the electrical signals to generate samples representing the gate location. The ultrasound data is coherent (i.e., maintained phase information), but may include incoherent data.

The Doppler processor 18 is a spectral Doppler processor. Other imaging detectors may be included, such as a B-mode and flow-mode processors. In one embodiment, the Doppler processor 18 is a digital signal processor or other device for applying a transform to the receive beam sample data. A sequence of transmit and receive events is performed over a period. A buffer or the memory 22 stores the receive beamformed data from each transmit and receive event. Any pulse repetition interval may be used for the transmit beams. Any number of transmit and receive events may be used for determining a spectrum, such as three or more. The Doppler processor 18 estimates a spectrum for the gate location. By applying a discrete or fast Fourier transform, or other transform, to the ultrasound samples for the same spatial location, the spectrum representing response from the location is determined. A histogram or data representing the energy level at different frequencies for the period of time to acquire the samples is obtained. Velocity may be determined from the frequency or frequency is used without conversion to velocity.

By repeating the process, the Doppler processor 18 may obtain different spectra for a given location at different times. Overlapping data may be used, such as calculating each spectrum with a moving window of selected ultrasound samples. Alternatively, each ultrasound sample is used for a single period and corresponding spectrum.

The processor 21 may be part of the Doppler processor 18 or a separate processor. The processor 21 or 18 or processors 18, 21 used for estimation or detection control the imaging and/or system 10. The processor 21 is a general processor, control processor, digital signal processor, application specific integrated circuit, field programmable gate array, graphics processing unit, analog circuit, digital circuit, combinations thereof or other now known or later developed device for processing.

The processor 21 is configured by hardware, software, or both to perform and/or cause performance of various acts, such as the acts discussed above for FIG. 2. The processor 21 is configured, as part of or in communication with the Doppler processor 18, to alter the spectrum or spectra based on a detected boundary. For example, a post-processing algorithm is run on a CPU, DSP or GPU to change the energy values after spectrum estimation and before generation of an image.

The processor 21 and/or processor 18 are configured to detect noise and signal regions of the spectrum. For example, the processor 21 detects the boundary between velocities associated with signal and velocities associated with noise. The Doppler processor 18 determines a clutter level, applies a thresholding function, receives a user entered trace, or otherwise determines characteristics for detecting the boundary. By determining a maximum and/or minimum velocity or other characteristic of each spectrum, velocities or frequencies associated with motion or flow may be distinguished from velocities or frequencies associated with noise.

The Doppler processor 18 and/or the processor 21 alter the energies or other characteristic of the spectrum based on the boundary. The noise and signal regions are weighted differently. The same type of weighting (e.g., same function) may be used, but with different weights. For example, at least some or all of the energies in the noise region of frequencies are attenuated, and/or at least some or all of the energies in the signal region of frequencies are enhanced (multiplied or increased). Different weighting functions may be used with the same or different weights. Variable scaling factors may be used for the noise, signal or both noise and signal weighting. Different types of alteration may be used. One region may be weighted while another is not or both are weighted with another difference. Any difference in weighting to separate the noise and signal information may be used.

Additional processes, such as filtering, interpolation, and/or scan conversion, may be provided by the Doppler processor 18, the processor 21, or another device. The altered spectrum or spectra are prepared and formatted for display. For example, the Doppler processor 18 generates display values as a function of the altered spectra estimated for the Doppler gate location. Display values include intensity or other values to be converted for display (e.g., red, green, blue values) or analog values generated to operate the display 20. The display values may indicate intensity, hue, color, brightness, or other pixel characteristic. For example, the color is assigned as a function of one characteristic of a spectrum and the brightness is a function of another spectrum characteristic or other information. The display values are generated for a spectral strip display.

The display 18 is a CRT, monitor, LCD, plasma screen, projector or other now known or later developed display for displaying an image responsive to the altered spectrum. For a grey scale spectral Doppler image, a range of velocities with each velocity modulated as a function of energy is provided as a function of time. A given spectrum indicates the velocity and energy information for a given time. The intensity of a given pixel or pixel region represents energy where velocity is provided on the vertical scale and time provided on the horizontal scale. Other image configurations may be provided, including colorized spectral Doppler images.

The memory 22 stores ultrasound samples for the range gate, estimated spectra, altered spectra, weights, scale functions, image data, or other information. The memory 22 may store information from any stage of processing or used for generating a display.

In one embodiment, the memory 22 is a non-transitory computer readable storage medium having stored therein data representing instructions executable by the programmed processor 18 for enhancing spectral Doppler imaging. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts, or tasks illustrated in the figures or described herein are executed in response to one or more sets of instructions stored in or on computer readable storage media. The functions, acts, or tasks are independent of the particular type of instructions set, storage media, processor, or processing strategy and may be performed by software, hardware, integrated circuits, firmware, micro code or the like, operating alone or in combination. Likewise, processing strategies may include multiprocessing, multitasking, parallel processing and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for enhancing spectral Doppler imaging, the method comprising:
estimating a spectrum for a Doppler gate location;
detecting a boundary between a noise region and a signal region of the spectrum;
altering, by a processor, the noise region, the signal region, or the noise and signal region of the spectrum, the altering being a function of the boundary; and
displaying an image, the image being a function of the altered spectrum.

2. The method of claim 1 wherein estimating the spectrum comprises applying a Fourier transform to a sequence of ultrasound signals representing the Doppler gate location, the spectrum comprising energy as a function of frequency.

3. The method of claim 1 wherein detecting the boundary comprises applying an energy threshold to the spectrum.

4. The method of claim 1 wherein detecting the boundary comprises identifying a velocity with an energy at a signal level adjacent to a velocity with an energy at a noise level.

5. The method of claim 1 wherein altering comprises attenuating energies in the noise region.

6. The method of claim 1 wherein altering comprises amplifying energies in the signal region.

7. The method of claim 1 wherein altering comprises scaling by a scaling factor.

8. The method of claim 1 wherein altering as a function of the boundary comprises altering differently for the noise region than the signal region.

9. The method of claim 1 wherein altering as a function of the boundary comprises setting a ramp function over a range of velocities, the range based on the boundary, the ramp function being for a variable scale factor applied to energy values.

10. The method of claim 1 wherein displaying comprises displaying a spectral strip including the altered spectrum for a first time.

11. The method of claim 1 further comprising repeating the estimating, detecting, and altering for an additional spectrum, the spectrum and the additional spectrum being for different times, and wherein displaying comprises displaying a spectral strip including the altered spectrum and an altered additional spectrum of the additional spectrum.

12. The method of claim 11 further comprising filtering the boundary across the different times prior to the altering and the repetition of the altering.

13. In a non-transitory computer readable storage medium having stored therein data representing instructions executable by a programmed processor for enhancing spectral Doppler imaging, the storage medium comprising instructions for:
locating an envelope in a spectral strip;
increasing a separation between signals of the spectral strip on different sides of the envelope; and
displaying the spectral strip with the signals having the increased separation.

14. The non-transitory computer readable storage medium of claim 13 wherein locating the envelope comprises, for each spectrum in the spectral strip, finding a boundary between a signal region and a noise region.

15. The non-transitory computer readable storage medium of claim 13 wherein increasing the separation comprises increasing the signals on a signal side of the envelope, decreasing the signals on a noise side of the envelope, or both.

16. The non-transitory computer readable storage medium of claim 13 wherein increasing comprises scaling the signals differently on the different sides.

17. The non-transitory computer readable storage medium of claim 13 wherein increasing comprises applying different weights to energies on the different sides, respectively, the weights for at least on of the sides including a ramp.

18. A system for enhancing spectral Doppler imaging, the system comprising:
a transmit beamformer operable to acoustic energy to a Doppler gate;
a receive beamformer operable to sample acoustic echoes from the Doppler gate and in response to the acoustic energy;
a spectral Doppler processor configured to estimate a spectrum from the samples of the acoustic echoes for the Doppler gate; and
a second processor configured to detect noise and signal regions of the spectrum and to weight the noise and signal regions of the spectrum differently.

19. The system of claim 18 wherein the second processor is configured to weight with a variable scaling factor across the noise, signal, or noise and signal regions.

20. The system of claim 18 wherein the second processor is configured to weight differently by attenuating energies in the noise region, enhancing energies in the signal region, or both.
